(19) United States
(12) Patent Application Publication
(10) Pub. No.: US 2018/0242655 A1
(43) Pub. Date: Aug. 30, 2018

(54) SMART FABRIC

(71) Applicants: Regents of the University of Minnesota, Minneapolis, MN (US); Mayo Foundation For Medical Education And Research, Rochester, MN (US)

(72) Inventors: Bradley Thomas Holschuh, North Oaks, MN (US); Lucy Elizabeth Dunne, Minneapolis, MN (US); Robert Michael Theodore Pettys-Baker, Minneapolis, MN (US); Nicholas Edward Schleif, Minneapolis, MN (US); Joonwoo Walter Lee, Minneapolis, MN (US); Mary Ellen Margaret Berglund, Minneapolis, MN (US); Sophia Utset-Ward, Minneapolis, MN (US); Michael J. Joyner, Rochester, MN (US); Bruce David Johnson, Rochester, MN (US); Kevin Lawrence Kelly, Rochester, MN (US); Christopher P. Johnson, Rochester, MN (US); Julia Claire Duvall, Minneapolis, MN (US); Rachael Granberry, Minneapolis, MN (US)

(21) Appl. No.: 15/905,372

(22) Filed: Feb. 26, 2018

Related U.S. Application Data
(60) Provisional application No. 62/463,356, filed on Feb. 24, 2017.

(51) Int. Cl. A41D 1/00; A41D 27/02; A41B 11/08; A61H 1/00; B32B 5/26

(52) U.S. Cl. CPC A41D 1/005; A61F 13/08; A41B 11/08; A61H 1/006; B32B 5/26; A41D 2400/44; A41D 2400/70; A41D 2400/32; A61H 2209/00; A61H 2205/108; A61H 2205/106; A61H 2201/5058; A61H 2201/1207; A61H 2201/5082; A41D 27/02

(57) ABSTRACT

Compressive garments described herein include an inner layer, a middle layer coupled to the inner layer, the middle layer comprising a plurality of circuits, each circuit of the plurality of circuits comprising a superelastic cable and a circuit breaker, and a outer layer removably coupled to at least one of the inner layer and the middle layer.

### RELATED APPLICATION

The present application claims the benefit of U.S. Provisional Application No. 62/463,356 filed Feb. 24, 2017, which is hereby incorporated herein in its entirety by reference.

### TECHNICAL FIELD

Embodiments described herein relate to compression garments.

### BACKGROUND

Compression garments or apparel have proved to be important in the consumer health and fitness industry as well as for clinical conditions. Compression garments are pieces of clothing, such as socks, pantyhose, sleeves, shirts, shorts, tights or underwear that are undersized (relative to the wearer), resulting in a static pressure created through elastic resistance. They are form-fitting garments that provide support, which can be especially useful for people who have to stand for long periods and people with poor circulation. Compression garments can have varying degrees of compression. The higher degrees require a doctor's prescription. Compression garments worn on the legs can help prevent deep vein thrombosis and reduce swelling, especially while traveling. For athletes, compression sportswear can also be worn during exercise to prevent chafing and rashes, and post-exercise to ease muscle stiffness and quicken recovery time.

Conventional compression garments are often made from elastic materials. Above certain levels of compression they can become uncomfortable, while undersized garments can be insufficiently compressive for other users. Undersized garments will rarely match a desired level of compression perfectly, unless they are tailored for a specific wearer. At any level of compression, conventional compression garments are difficult to don or doff, especially for users with limited flexibility or strength.

Among the alternatives to elastic compression stockings are inflatable compression sleeves. These sleeves provide a more precise quantity of compression, which can be controlled by adjusting the magnitude and timing of inflation. However, inflation-based compression garments typically require the wearer to remain stationary, are highly immobile, and must be tethered to the inflation source. Neither design, whether undersized compression garments or inflated compression garments, offers a solution to the consumer that is simultaneously low profile, mobile, and controllable.

### SUMMARY

In an embodiment, a compression garment includes an inner layer, a middle layer, and an outer layer. The middle layer can be coupled to the inner layer. The middle layer includes a plurality of circuits, and each circuit includes a superelastic cable and a circuit breaker. The outer layer can be removably coupled to at least one of the inner layer and the middle layer.

In another embodiment, a system of compression garments includes a left calf portion, a right calf portion, a left thigh portion, and a right thigh portion. Each of the portions includes an inner layer, a middle layer, and an outer layer. Each of the middle layers is coupled to the corresponding inner layer. Each middle layer includes a first plurality of circuits, each of which includes both a superelastic cable and a circuit breaker. The outer layer can be removably coupled to at least one of the inner layer and the middle layer.

The details of one or more implementations are set forth in the accompanying drawings and the description below. The above summary is not intended to describe each illustrated embodiment or every implementation of the subject matter hereof. The figures and the detailed description that follow more particularly exemplify various embodiments.

### DESCRIPTION OF DRAWINGS

Subject matter hereof may be more completely understood in consideration of the following detailed description of various embodiments in connection with the accompanying figures, in which:

FIGS. 1A-1C are front, side, and back views of a compressive garment, respectively, according to an embodiment.

FIGS. 2A-2C are various views of a compressive garment according to an embodiment.

FIG. 3 depict an open view of the compressive garment of FIGS. 1A-1C and 2A-2C.

FIG. 4A is a cross-sectional view of a longitudinal panel in a relaxed configuration according to an embodiment.

FIG. 4B is a cross-sectional view of a longitudinal panel in an activated configuration according to an embodiment.

FIGS. 5A and 5B are front and side views of a set of support garments according to an embodiment.

FIG. 6 is a cross-sectional side view of a compressive garment for providing non-uniform compression according to an embodiment.

FIGS. 7A and 7B are cross-sectional side views of a compressive garment having segmented activation according to an embodiment.

FIG. 8 shows an example compressive vest garment according to an embodiment.

FIG. 9 is a flow diagram of an example process for using a compressive garment according to an embodiment.

FIG. 10 is a schematic diagram of an example of a generic computer system.

FIG. 11 is a plan view of a three-layer garment according to an embodiment.

FIG. 12 is a perspective view of a system including a left calf garment, a right calf garment, a left thigh garment, and a right thigh garment, according to an embodiment.

FIG. 13 is an exploded view of an inner layer, a middle layer, and an outer layer of a garment according to an embodiment.

FIG. 14 is a circuit diagram of a compression garment according to an embodiment.

FIGS. 15A and 15B depict closed and open versions of a tension-sensitive latch circuit breaker according to an embodiment.

FIG. 16 is a graph of tension and resistance at a tension-sensitive latch circuit breaker according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

This document describes systems and techniques for applying variable and controllable amounts of compression to various body parts through the use of actively compressible garments. In general, the compressive garments have tubular shapes sized to accommodate various, generally cylindrically-shaped body parts (e.g., calf, thigh, upper arm, forearm, torso). These garments generally have a manually-adjustable portion (e.g., a lacing) to ease the process of donning the garment and sizing it to the wearer at a first (e.g., low) level of compression, and an actively adjustable portion (e.g., a shape memory material) that can be controllably actuated. When the actively adjustable portion is actuated, the diameter of the tubular shape of the garment is reduced, providing an increase in compression about the body part inside. By de-actuating the actively adjustable portion, the garment can relax to its original, larger diameter and reduce the amount of compression about the body part.

This document describes examples of the use of “smart fabrics” with embedded shape-changing materials that can provide controllable compression without the need for the bulky inflation systems generally associated with inflatable compression systems. The use of embedded shape-changing materials allows for more specific dynamic control over the degree of compression, the timing of compression, and allow for graded compression (e.g., peripherally to more centrally guided pressure) without overly encumbering the wearer. Such designs can provide a slim, low-profile form factor that is easy to don and remove, and provides dynamic control of compression.

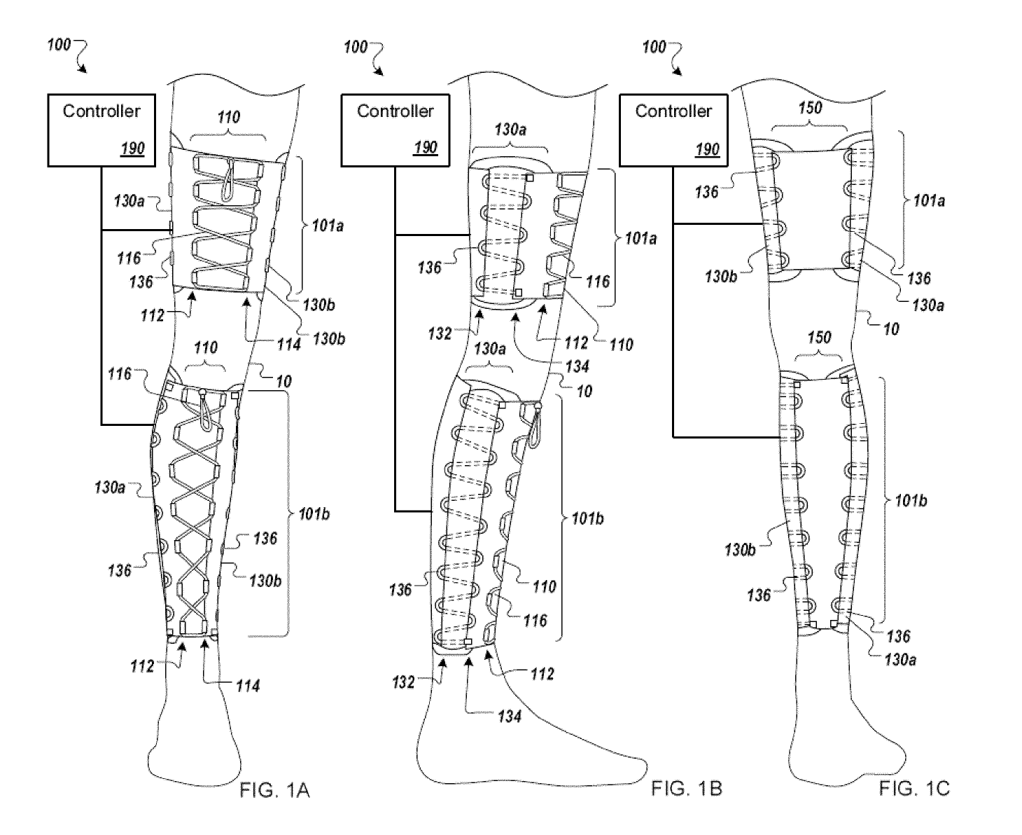

FIGS. 1A-1C are rear, right, and front side views, respectively, of an example compressive garment 100. The compressive garment 100 is configured to provide compression to a human leg 10, and includes a thigh portion 101a and a calf portion 101b. The thigh portion 101a and the calf portion 101b are both generally tubular in shape and sized to accommodate an approximately cylindrical-shaped body part, such as the leg 10. Although the example compressive garment 100 is shown and described as being configured to be worn on a human leg, the compressive garment 100 and the other embodiments that will be described below can be adapted to be worn on other generally cylindrical human body parts (e.g., arm, finger, torso), as well as non-human body parts (e.g., veterinary applications). Furthermore, which the compressive garment 100 is described as having two portions (e.g., the thigh portion 101a and the calf portion 101b), in other embodiments compressive garments can include a single portion, or any appropriate number of portions (e.g., a finger application may have three portions: a first for the distal phalanges, a second for the intermediate phalanges, and a third for the proximal phalanges).

Each of the portions 101a and 101b is a generally tubular compressive garment that includes a passive longitudinal panel 110, an active longitudinal panel 130a, an active longitudinal panel 130b, and a connective longitudinal panel 150. The panels 110, 130a, 130b, and 150 and interconnected to form a tube, in which the passive longitudinal panel 110 is connected at one lengthwise edge to a lengthwise edge of the active longitudinal panel 130a, the opposite lengthwise edge of the active longitudinal panel 130a is connected to a lengthwise longitudinal panel of the connective longitudinal panel 150, an opposite lengthwise longitudinal panel of the connective longitudinal panel 150 is connected to a lengthwise edge of the active longitudinal panel 130b, and an opposite lengthwise edge of the active longitudinal panel 130b is connected back to the opposite lengthwise edge of the passive longitudinal panel 110.

Each of the passive longitudinal panels 110 includes a panel portion 112, a panel portion 114, and an adjustable fastener 116. The panel portion 112 is connected to the active longitudinal panel 130a, and the panel portion 114 is connected to the active longitudinal panel 130b. The adjustable fastener 116 is configurable to connect the panel portion 112 to the panel portion 114 at a manually selected and adjustable circumferential spacing.

In the illustrated example, the adjustable fastener 116 is a lacing that can be drawn to pull the panel portion 112 and the panel portion 114 closer to each other in order to adjust the circumference of the respective portion 101a or 101b to fit the wearer, and then tied in order to maintain the adjusted sizing. In other embodiments, some of which will be discussed below, other adjustable and non-adjustable fasteners may be used (e.g., zippers, hook and loop fasteners, buckles, hooks and eyes, rings and slides). In some implementations, multiple adjustable fasteners such as the adjustable fastener 116 may be used. For example, multiples of the adjustable fastener 116 can be arranged longitudinally along the longitudinal panels 110, and each adjustable fasteners 116 can be independently adjusted to provide differing amounts of initial compression at different positions long the leg 10.

Each of the active longitudinal panels 130a and 130b includes a panel portion 132, a panel portion 134, and an adjustable fastener 136. One of the panel portions 132 is connected to the connective longitudinal panel 150, and the other of the panel portions 134 is connected to the passive longitudinal panel 110. The adjustable fastener 136 is configurable to connect the panel portion 132 to the panel portion 134.

The adjustable fasteners 136 each at least partly includes a shape-memory actuator configurable to connect the panel portion 132 to the panel portion 134 at an adjustable circumferential spacing. Each of the adjustable fasteners 136 is a lacing in which the shape-memory actuator is laced between the panel portion 132 and the panelportion 134. The adjustable circumferential spacing generally ranges from relaxed circumferential spacing based on a deformed shape-memory configuration of the shape-memory actuator of the adjustable fastener 136, and a compressed circumferential spacing, different from and generally smaller than the relaxed adjustable circumferential spacing, based on a recovered shape-memory configuration of the shape-memory actuator.

The shape-memory actuator of each of the adjustable fasteners 136 is formed as a helical tension spring formed of a metal alloy comprising nickel and titanium. The adjustable fasteners 136 are configured to have a first length in the deformed shape-memory configuration and to have a second length, different from the first length, in the recovered shape-memory configuration The metal alloy is configured to be at least partly transitioned from the second length to the first length by tension between the panel 132 and the panel 134, and the metal alloy is configured to be at least partly transitioned from the first length to the second length by applying a predetermined temperature to the metal alloy. In some embodiments, the shape-memory actuator can be made of other shape memory metal alloys or other shape-memory materials (e.g., shape-memory polymers, photoreactive materials).

The compressive garment includes a controller 190. The controller 190 is operatively connected to the adjustable fasteners 136 of the thigh portion 101a and the calf portion 101b. The controller 190 is configured to controllably actuate and de-actuate the adjustable fasteners 136. In the illustrated example, the controller 190 can controllably apply heat to activate recovery of the shape-memory actuator(s) of the adjustable fasteners 136. For example, the controller 190 can pass an electrical current through the metal alloy of the adjustable fastener 136 to create heat that can activate recovery of the shape-memory metal alloy. In another example, the controller 190 can power up a heating element that is on or near the adjustable fastener 136 to trigger the shape recovery. In some embodiments, the recovery process may be triggered by other techniques, such as by exposing a photoreactive shape-memory material to an appropriate light source (e.g., infrared, ultraviolet).

In some embodiments, the shape-memory actuator of each of the adjustable fasteners 136 can be tensioned to the first length, and at least partly return to the second length, which is shorter than the first length, when exposed to the predetermined temperature. For example, tension between the panels 132 and 134 (e.g., caused by tightening the adjustable fastener 116) can stretch the lacing of the adjustable fastener 136, distorting the metal alloy. Heat can be applied to the lacing to cause the shape-memory alloy to recover toward its original, shorter length, thereby drawing the panels 132 and 134 toward each other and controllably tightening the compression provided by the garment 100.

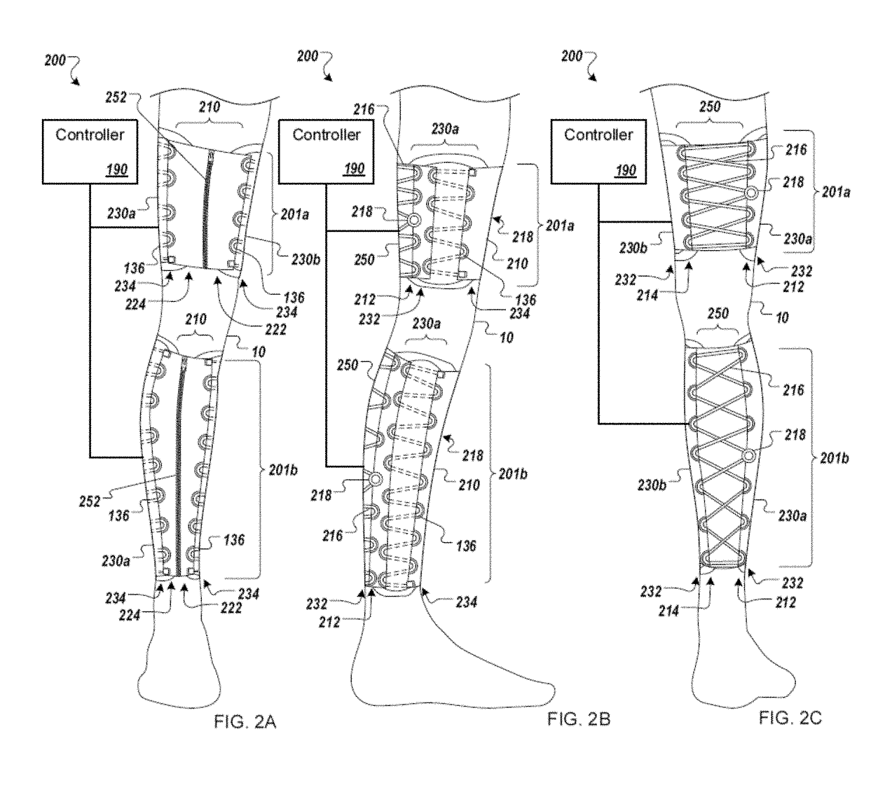

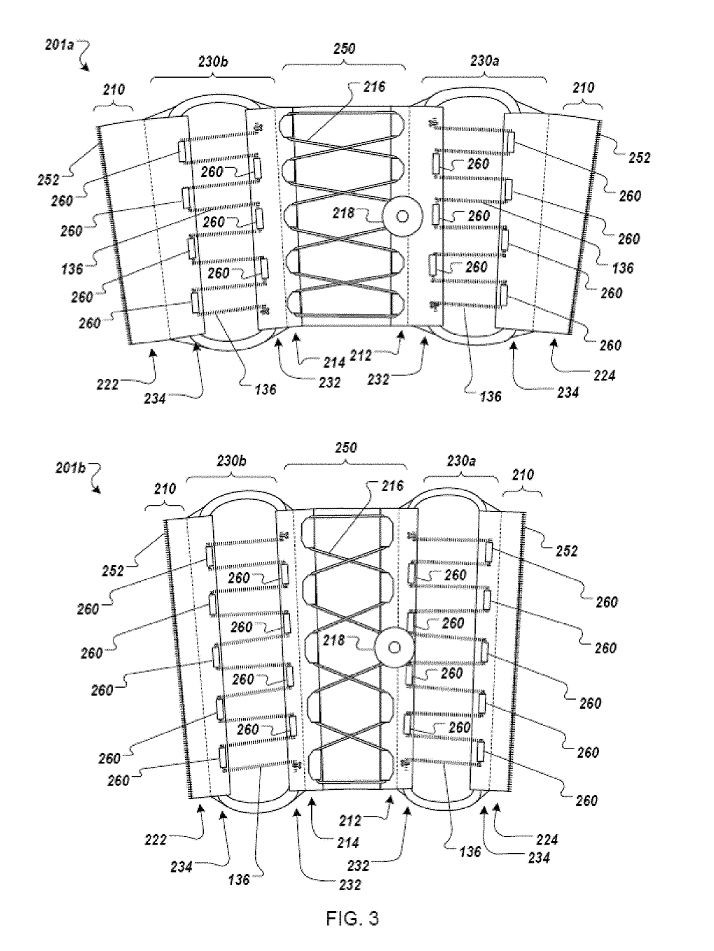

FIGS. 2A-2C are rear, right, and front side views, respectively, of another example compressive garment 200. FIG. 3 is an additional view of the example compressive garment 200 of FIGS. 2A-2C, with the garment 200 opened and removed from the leg 10. Whereas FIGS. 2A-2C show an example of the compressive garment 200 donned upon the leg 10, FIG. 3 shows the compressive garment 200 in an unworn state.

The compressive garment 200 is substantially similar to the example compressive garment 100 of FIGS. 1A-1C, being configured to provide compression to a human leg 10, and including a thigh portion 201a and a calf portion 201b. Similar to the thigh portion 101a and the calf portion 101b, the thigh portion 201a and the calf portion 201b are both generally tubular in shape and sized to accommodate an approximately cylindrical-shaped body part, such as the leg 10.

Each of the portions 101a and 101b is a generally tubular compressive garment that includes a passive longitudinal panel 210, an active longitudinal panel 230a, an active longitudinal panel 230b, and a connective longitudinal panel 250. The panels 210, 230a, 230b, and 250 and interconnected to form a tube, in which the passive longitudinal panel 210 is connected at one lengthwise edge to a lengthwise edge of the active longitudinal panel 230a, the opposite lengthwise edge of the active longitudinal panel 230a is connected to a lengthwise longitudinal panel of the connective longitudinal panel 250, an opposite lengthwise longitudinal panel of the connective longitudinal panel 250 is connected to a lengthwise edge of the active longitudinal panel 230b, and an opposite lengthwise edge of the active longitudinal panel 230b is connected back to the opposite lengthwise edge of the passive longitudinal panel 210.

The active longitudinal panels 230a and 230b are substantially similar to the example longitudinal panels 130a and 130b of the compressive garment 100. For example, the active longitudinal panels 230a and 230b include a lacing made up of the adjustable fasteners 136 that can be controllably lengthened and shortened through mechanical distortion caused by pulling (e.g., tensioning) the longitudinal panels 230a, 230b, and through active recovery caused by heating that is applied by the controller 190. Each of the active longitudinal panels 230a and 230b includes a panel portion 232, a panel portion 234. One of the panel portions 232 is connected to the connective longitudinal panel 250, and the other of the panel portions 234 is connected to the passive longitudinal panel 210. The adjustable fastener 136 is configurable to connect the panel portion 232 to the panel portion 234.

Each of the passive longitudinal panels 250 includes a panel portion 212, a panel portion 214, and an adjustable fastener 216. The panel portion 212 is connected to the active longitudinal panel 230a, and the panel portion 214 is connected to the active longitudinal panel 230b. The adjustable fastener 216 is configurable to connect the panel portion 212 to the panel portion 214 at a manually selected and adjustable circumferential spacing. In the illustrated example, the adjustable fastener 216 includes a ratcheting, rotary-type adjustment mechanism 218.

In the illustrated example, the adjustable fastener 216 is a lacing that can be drawn to pull the panel portion 212 and the panel portion 214 closer to each other. The adjustment mechanism 218 can be rotated to lengthen and shorten the adjustable fastener 216 to tighten and loosen the lacing of the passive longitudinal panels 210 in order to adjust the circumference of the respective portion 201a or 201b to fit the wearer. The adjustment mechanism 218 can then be reversibly secured to maintain the selected sizing.

Each of the connective panels 210 is substantially similar to the connective panels 150 of the example compressive garment 100, but also includes a panel portion 222, a panel portion 224, and a separable fastener 252. The panel portion 224 is connected to the active longitudinal panel 230a, and the panel portion 222 is connected to the active longitudinal panel 230b. In the illustrated example, the separable fastener 252 is a zipper, but in other embodiments the separable fastener 252 can be any other type of fastener that can be separated and rejoined (e.g., hook and loop fasteners, buckles, hooks and eyes, rings and slides) to connect the panel portion 222 to the panel portion 224.

Referring now to FIG. 3, the compressive garment 200 is shown in an unworn state. For example, the separable fastener 252 (e.g., zipper) is unfastened (e.g., unzipped) such that the generally tubular shape of the compressive garment 200 as worn is flattened into the configuration shown in FIG. 3. In general, the compressive garment 200 can be wrapped around the leg 10 such that the panel portions 222 and 224 meet, and the separable fastener 252 can be fastened to reform the generally tubular shape shown in FIGS. 2A-2C. In some implementations, the thigh portion 101a and the calf portion 101b can be donned and sized by adjusting the adjustable fastener 216. Once sized, the compressive garment can be quickly removed and re-donned at the adjusted size by separating and rejoining the separable fastener 252 (e.g., zipping the garment 200 on and off as needed by the wearer).

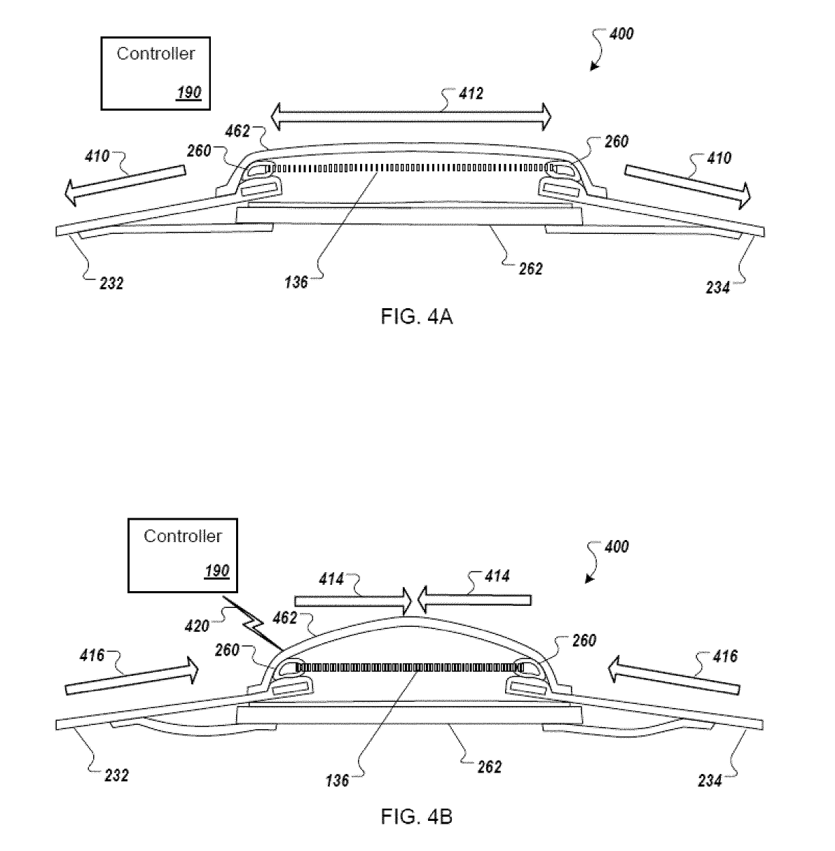

From the view shown in FIG. 3, it can be seen that the lacings of the active longitudinal panels 230a and 230b also include a collection of thermally insulating boning portions 260 and a thermally insulating liner 262. The thermally insulating boning portions 260 and the thermally insulating layer 262 are configured to withstand the predetermined temperature of actuation of the shape-memory actuator of the adjustable fastener 136. For example, to recover the length of the adjustable fastener 136, the adjustable fastener 136 may be heated. These temperatures, however, may be high enough to become damaging to the panel portions 212, 214, 222, or 224, or may become uncomfortable or harmful to the wearer. The thermally insulating boning portions 260 thermally isolate the panel portions 212, 214, 222, or 224 from this heat. The thermally insulating layer 262 is positioned between the adjustable fastener 136 and the wearer's skin to thermally isolate the wearer from this heat. In some embodiments, the thermally insulating layer 262 may also prevent the adjustable fastener 136 from chafing against the wearer's skin. As will be discussed in the description of FIGS. 4A and 4B, another thermally insulating layer may also be included to cover the outside of the adjustable fasteners 136, to protect and insulate the adjustable fasteners 136.

FIG. 4A is a cross-sectional view of an example longitudinal panel 400 in a relaxed configuration. FIG. 4B is a cross-sectional view of the example longitudinal panel 400 in an activated configuration. In some embodiments, the longitudinal panel 400 can be any of the example active longitudinal panels 130a, 130b, 230a, and 230b of FIGS. 1A-3. In the illustrated example, the adjustable fastener 136, the panel portion 232, the panel portion 234, the boning portions 260, and the thermally insulating layer 262 are shown.

The adjustable fastener 136 at least partly includes a shape-memory metal alloy that is arranged as a helical tension spring. The spring is configured to be tensioned to a longer, distorted length, and is configured to at least partly return to a shorter, recovered length when exposed to a predetermined temperature.

As discussed earlier, the thermally insulating layer 262 is positioned between the adjustable fastener 136 and the wearer's skin. The thermally insulating layer 262 thermally isolates the wearer from heat applied to or generated by the adjustable fastener 136 during shape-memory recovery. The thermally insulating layer 262 also prevents the adjustable fastener 136 from chafing against the wearer's skin and from becoming damaged though such contact.

The longitudinal panel 400 also includes a cross-fastener 462, in some embodiments. The cross-fastener 462 at least partly covers the adjustable fastener 136. The cross-fastener 462 connects one of the panel portions 232 to the other of the panel portions 234.

FIG. 4A shows the example longitudinal panel 400 in a distorted (e.g., or relaxed) configuration. Circumferential tension on the longitudinal panel 400, represented by the arrows 410, distorts and deforms the adjustable fastener 136 into an extended length, as represented by the arrow 412.

The cross-fastener 462 substantially limits the maximum spacing by which the panel portion 232 to the panel portion 234 can be separated, and thereby limit the maximum distortion of the adjustable fastener 136. For example, some embodiments of the adjustable fastener 136 may become irrecoverably distorted when distorted beyond a predetermined distortion limit. In the illustrated example, as the panel portion 232 and the panel portion 234 separate, stretching the adjustable fastener 136 and the cross-fastener 462. Eventually the cross-fastener 462 becomes taut, thereby limiting the separation of the panel portion 232 from the panel portion 234 and limiting further deformation of the adjustable fastener 136.

In some embodiments, the cross-fastener 462 can protect the adjustable fastener 136, for example, to prevent the adjustable fastener 136 from snagging on outer layers of clothing (e.g., when worn under a shirt or pants) or to smooth the appearance of the longitudinal panel 400 (e.g., to make it less visibly noticeable when worn under clothing). In some embodiments, the cross-fastener 462 can be a thermally insulating layer configured to retain heat applied during or generated by the shape-memory recovery process (e.g., to reduce the amount of electrical energy needed for recovery).

Referring now to FIG. 4B, the example longitudinal panel 400 is shown in a recovered or recovering configuration, as indicated by the arrows 414 and 416. The controller 190 provides an actuation signal 420 (e.g., applying a heating current to the adjustable fastener 136) which causes the adjustable fastener 136 to recover to the shorter length, as represented by the arrows 414. Recovery activation of the adjustable fastener 136 creates circumferential tension on the longitudinal panel 400, represented by the arrows 416, reducing the overall circumference of the garment and increasing compression to the body part about which the garment is being worn.

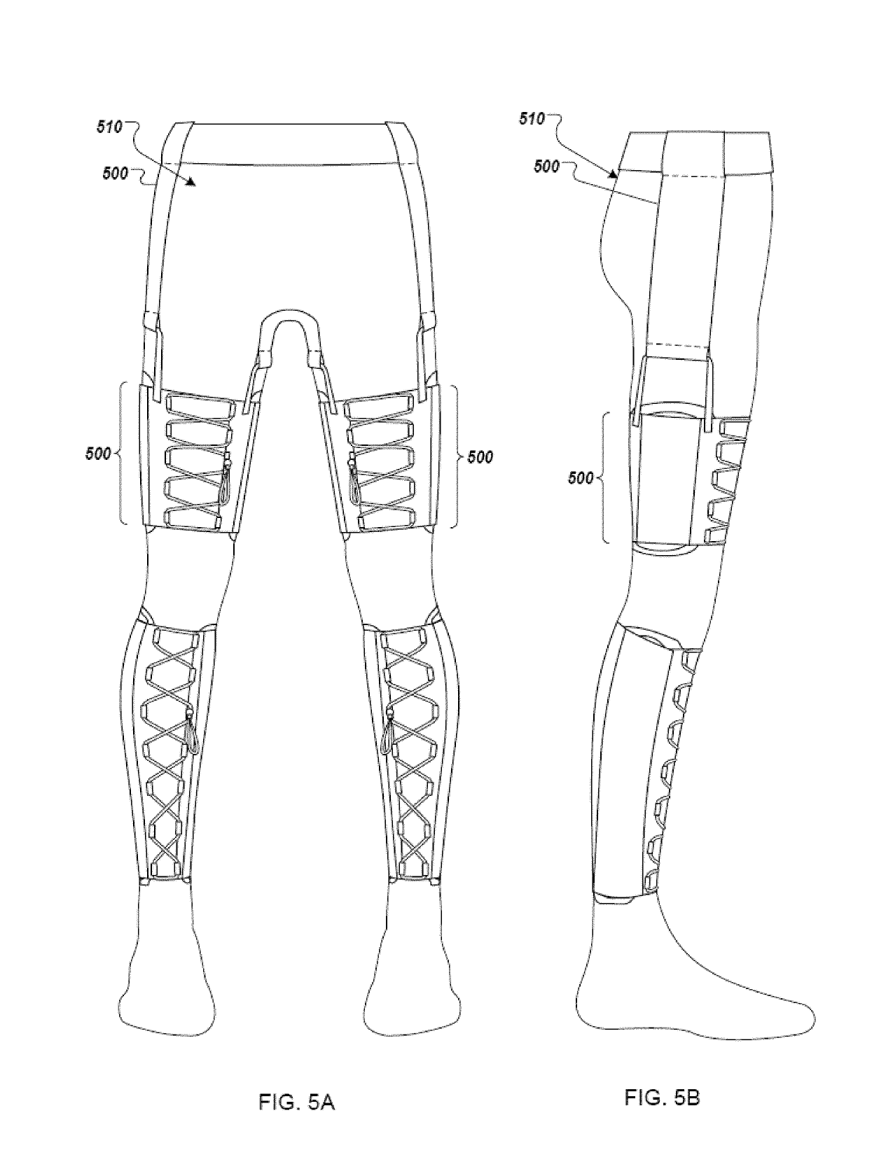

FIGS. 5A and 5B are front and side views of an example support garment 500 for an example compressive garment 510. In some embodiments, the compressive garment 500 can be the example thigh portion 101a or 201a of FIGS. 1A-2C.

The support garment 500 is worn about the wearer's hips 510. The support garment 500 is removably suspended from the compressive garment 510 to provide vertical support and maintain the position of the compressive garment 510 on the wearer. For example, the human thigh has a shape that generally resembles an inverted conic section that tapers downwards. Contraction and relaxation of the compressive garment 510, movement of the wearer, gravity, and other factors can cause the compressive garment to creep or slip downwards. The support garment 500 acts as a garter or suspender by transferring some of the downward force to the wearer's hips.

In some embodiments, the support garment 500 can have other forms. For example, the support garment 500 can be formed as a pair of suspenders that extend form the compressive garment to the wearer's shoulders. In some embodiments, the support garment 500 can be adapted for use with the example calf portions 101b and 201b. For example, the support garment 500 can be configured similar to a men's stocking garter, in which the calf portion is removably connected to a band that is worn about an upper, thinner portion of the calf.

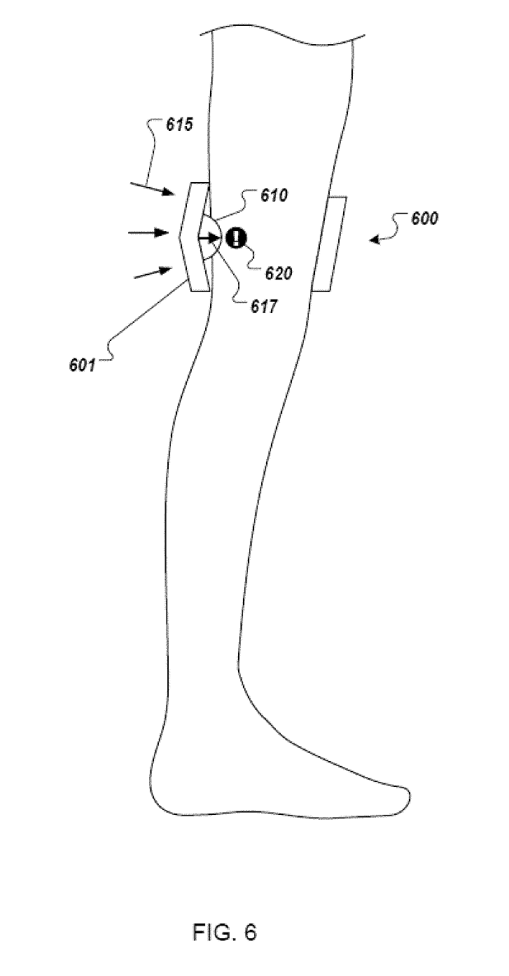

FIG. 6 is a cross-sectional side view of a compressive garment 600 for providing non-uniform compression. Generally speaking, the example compressive garments 100 and 200 provide compression that is substantially uniform along the longitudinal lengths of the example thigh portions 101a, 201a and/or the calf portions 101b, 201b. In some embodiments, however, non-uniform or targeted compression may be desired.

The compressive garment 600 includes a thigh portion 601. In some embodiments, the thigh portion 601 can be the example thigh portions 101a, 201a. The compressive garment 600 is actively compressible (e.g., through use of one or more of the active panel portions 130a, 130b, 230a, 230b, 400 described above). The compressive garment also includes a compression member 610. The compression member 610 is firm object arranged between the thigh portion 601 and a compression target area on the wearer's body, as represented by 620.

In use, as the thigh portion 601 contracts, the compression member 610 redirects the generally uniform, radially inward compression forces, represented by the arrows 615, and redirects and focuses the forces 615 toward the target area 620, as represented by the arrow 617.

In some implementations, one or more compression members such as the compression member 610 can be arranged to contact the wearer at any appropriate longitudinal or radial position. For example, multiples of the compression member 610 having different sizes may be arranged about a selected longitudinal position on the body to provide non-uniform circumferential compression at the selected position. In some implementations, non-uniform compression may be used to massage, compress, or stimulate a selected location on the wearer's body. For example, the compression member 610 may be used for performing acupressure. In another example, the compression member 610 may be located near an artery, and the compressive garment 600 may be activated to apply pressure to reduce blood flow through the artery.

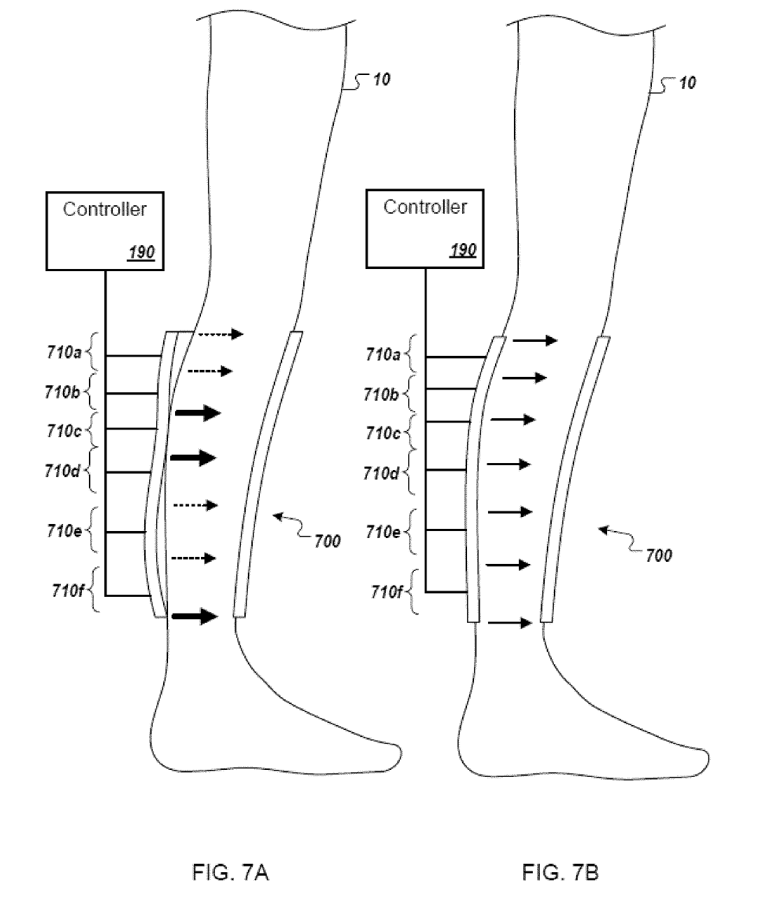

FIGS. 7A and 7B are cross-sectional side views of a compressive garment 700 having non-uniform activation. As discussed above, in some embodiments non-uniform or targeted compression may be desired. In some embodiments, the compressive garment 700 can be a modification of the example calf portion 101b or 201b of FIGS. 1A-3.

The compressive garment 700 includes an active longitudinal panel (not shown). The active longitudinal panel is configured with a collection of substantially independent active subportions 710a-710f arranged longitudinally along the tubular shape of the compressive garment 700. For example, the active longitudinal panel can include an adjustable fastener made up of multiple, separate shape-memory subportions that can be independently actuated and relaxed by the controller 190. In another example, the active longitudinal panel can include multiple, separate adjustable fasteners each having a shape-memory portion, wherein each of the adjustable fasteners can be independently actuated and relaxed by the controller 190.

Referring to FIG. 7A, the active subportions 710c, 710d, and 710f are in contact with the leg 10, while the active subportions 710a, 710b, and 710e are not. In such an example, if the entire compressive garment 700 (e.g., all of the active subportions 710a-710f) where to be actuated uniformly, the regions of the leg 10 near the active subportions 710c, 710d, and 710f may experience compression sooner or to a greater extent than the portions of the leg 10 near the active subportions 710a, 710b, and 710e. However, in the example of the compressive garment 700, the controller 190 can activate each of the active subportions 710a-710f independently.

Referring now to FIG. 7B, the controller 190 has activated the active subportions 710a, 710b, and 710e sooner or to a greater degree than the active subportions 710c, 710d, and 710f. As such, the compressive garment 700 can provide non-uniform actuation to compress the leg 10. In some implementations, non-uniform actuation may be performed to provide uniform compression along a non-uniformly shaped body part. In some implementations, non-uniform actuation may be performed to provide targeted compression at points along the body part upon which the compressive garment 700 is worn. For example, the controller 190 may be configured to actuate a single one of the active subportions 710a-710f to perform a function similar to the example compressive garment 600 of FIG. 6.

In some implementations, non-uniform activation may be used to massage, compress, or stimulate a selected location on the wearer's body. For example, the active subportions 710a-710f may be activated in predetermined patterns (e.g., bottom to top) to provide a massaging or fluid pumping action (e.g., to urge the redistribution of fluids in the extremities). In another example, one or more of the active subportions 710a-710f may be activated in response to an injury to apply pressure to an artery in an attempt to reduce blood loss (e.g., an automated tourniquet).

In some embodiments, the controller 190 may be configured receive biofeedback signals and to provide uniform or non-uniform activation in response to such signals. For example, the controller 190 may receive electrocardiogram signals and synchronize a patterned actuation of the active subportions 710a-710f to compress the leg 10 to provide a supplemental blood-pumping action for the wearer.

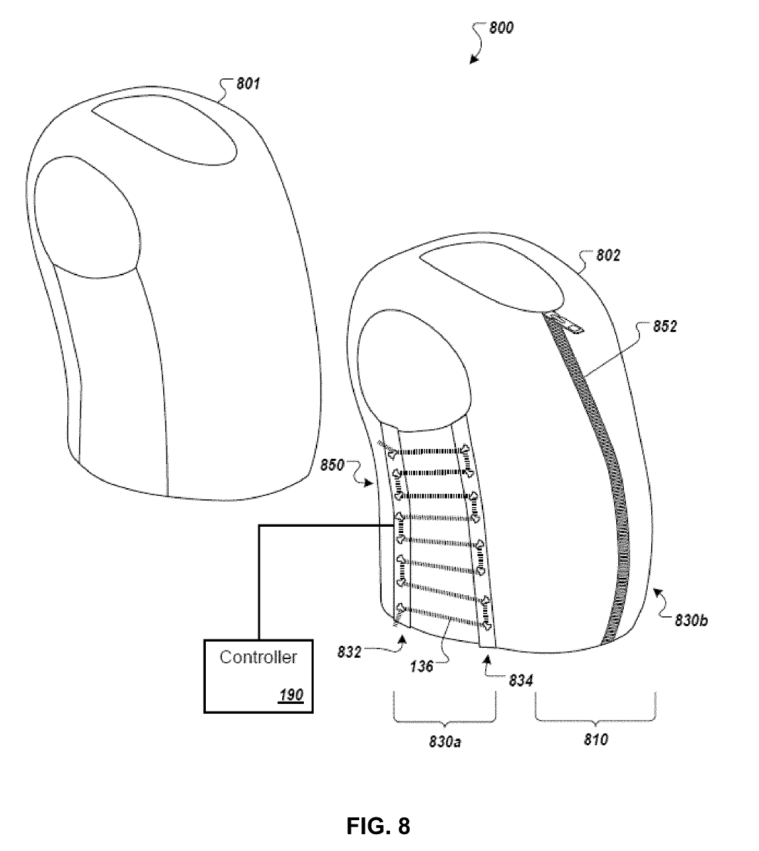

FIG. 8 shows an example compressive vest garment 800. Generally speaking, the compressive vest garment 800 is an adaptation of the example thigh portion 201a or the example calf portion 201b, modified to be worn about the chest and torso. The compressive vest garment includes a liner portion 801 and a compressive shell portion 802. In use, the liner portion 801 is worn under the compressive shell portion 802.

The compressive shell portion 802 includes a passive longitudinal panel 810, an active longitudinal panel 830a, an active longitudinal panel 830b (not directly visible in this view), and a passive longitudinal panel 850 (not directly visible in this view).

The passive longitudinal panel 850 is arranged similar to the example passive longitudinal panels 150 and 250 of FIGS. 1A-3. For example, the passive longitudinal panel 850 can include a lacing that is manually adjustable to size the compressive shell portion 802 to an individual wearer and set an initial, minimum amount of compression about the wearer's chest.

The passive longitudinal panel 810 includes a separable fastener 852, and is arranged similar to the example passive longitudinal panel 250 of FIGS. 2A-3. For example, the separable fastener 852 can be separated (e.g., unzipped) to allow the compressive shell portion 802 to be donned like a vest, and the rejoined (e.g., zipped) to reform the compressive shell portion 802 as a generally tubular garment about the wearer's torso.

The active longitudinal panels 830a and 830b each include the adjustable fastener 136. The controller 190 can actuate the adjustable fasteners 136 to controllably draw a panel portion 832 and a panel portion 834 closer together, thereby reducing the circumference of the compressive shell portion 802 and applying compression to the wearer's torso.

In some implementations, the compressive vest garment 800 can be used in therapeutic applications. For example, individuals with Autism Spectrum Disorder (ASD) and Attention Deficit and Hyperactivity Disorder (ADHD) often seek out deep touch pressure (DTP) therapies. Previously, weighted blankets, weighted vests, and other weighted wearable garments have been used to provide DTP, but each suffers from significant limitations (e.g., weight, bulk, difficulty in donning the garment upon a reluctant patient). The compressive vest garment 800, however, can be made without much of the weight and bulk of weighted garments, and as such can be donned much more easily. The shape-memory materials of the adjustable fasteners 136 can be actuated by the controller 190 to contract when heated to create a deep pressure vest that can constrict on command, while being simultaneously low profile and adjustable. In some embodiments, the controller 190 can be controlled remotely, allowing wearer self-adjustment and enabling the patient's parent, guardian, or occupational therapist (OT) to give a comforting “hug” from potentially anywhere in the world.

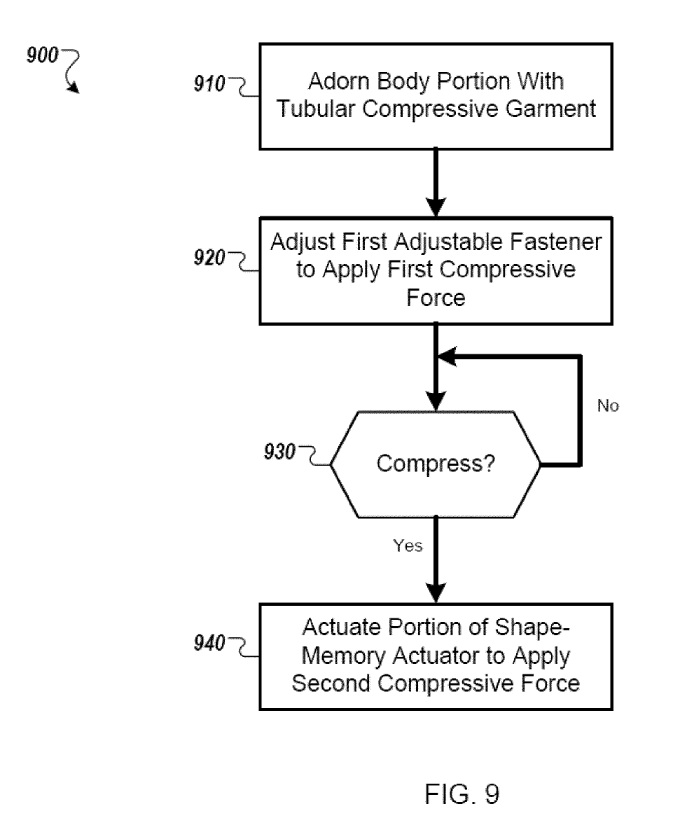

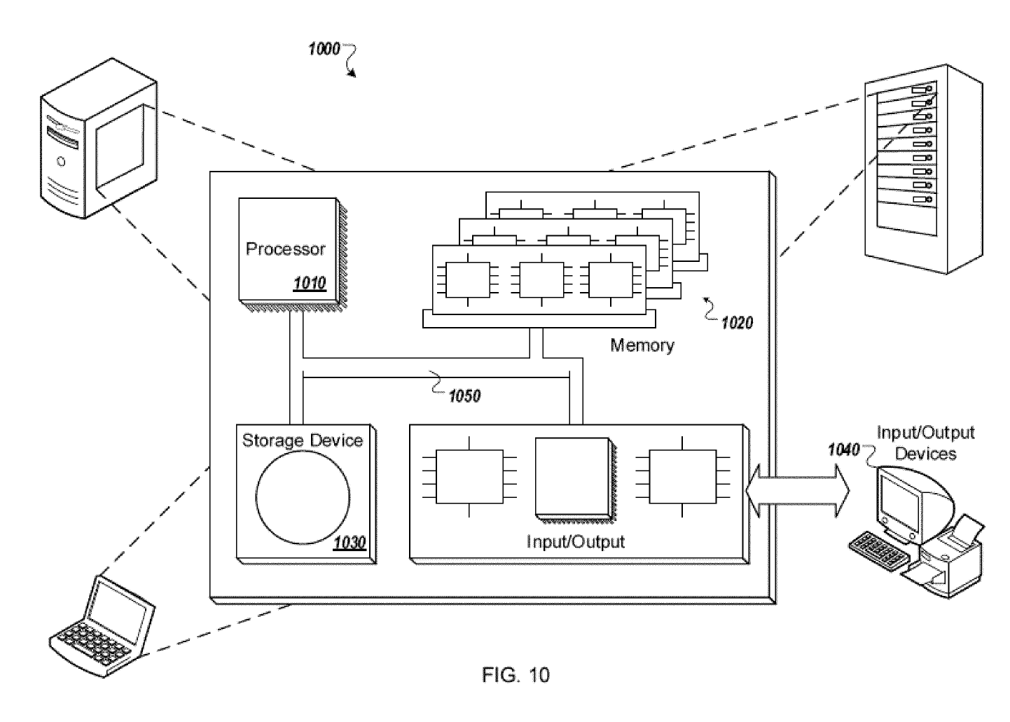

FIG. 9 is a flow diagram of an example process 900 for using a compressive garment. In some implementations, the process 900 can be used with any of the example compressive garments 100, 200, 500, 600, 700, and 800, and the example longitudinal panel 400 of FIGS. 1A-8. For example, the process 900 can be performed by the controller 190.

At 910 a body portion is adorned with a tubular compressive garment. The compressive garment includes a first panel portion, a second panel portion, and a first adjustable fastener configurable to connect the first panel portion to the second panel portion, a second longitudinal panel having a third panel portion, a fourth panel portion, and a second adjustable fastener comprising a shape-memory actuator configurable to connect the third panel portion to the fourth panel portion. For example, the compressive garment 100 can be adorned upon the leg 10.

At 920, the first adjustable fastener is adjusted to apply a first compressive force about the body portion. In some implementations, the first adjustable fastener can be adjusted to draw the first panel portion toward the second panel portion at a selected circumferential spacing. For example, the adjustable fastener 116 of the passive longitudinal panel 110 can be adjusted to draw the panel portion 112 to the panel portion 114 at a manually selected and adjustable circumferential spacing. By adjusting the spacing of the panel portions 112 and 114, the thigh portion 101a and/or the calf portion 101b can be tightened to provide an initial (e.g., minimum) amount of compression to the leg 10.

In some implementations, the process 900 can include drawing the third panel portion and the fourth panel portion apart to a first adjustable circumferential spacing, and deforming the shape-memory actuator to a deformed shape-memory configuration. For example, as the adjustable fastener 116 is tightened, the panel portions 132 and 134 can be drawn apart, stretching the adjustable fastener 136 away from its shorter, recovered shape.

In some implementations, the process 900 can include limiting, by at least one cross-fastener connecting the third panel portion to the fourth panel portion, drawing of the third panel portion from the fourth panel portion to no greater than the first adjustable circumferential spacing. For example, the cross-fastener 462 connects the panel portion 232 to the panel portion 234. The cross-fastener 462 can substantially limit the maximum spacing by which the panel portion 232 to the panel portion 234 can be separated, and can thereby limit the maximum distortion of the adjustable fastener 136.

If at 930 no additional compression is to be applied, then the process 900 waits. However, if at 930 it is determined that additional compression is to be applied, then the process 900 continues at 940.

At 940, at least a portion of the shape-memory actuator is actuated to apply a second compressive force, greater than the first compressive force, about the body portion. For example, the controller 190 can respond to an input (e.g., a user button press, a timer event, a biofeedback signal) and respond by actuating the adjustable fastener 136 to cause the adjustable fastener 136 to at least partly recover toward a shorter, recovered shape, thereby shrinking the circumference of the compressive garment 100 and increasing the amount of compression about the leg 10.

In some implementations, actuating at least a portion of the shape-memory actuator to apply a second compressive force, greater than the first compressive force, about the body portion can include drawing, by the shape-memory actuator, the third panel toward the fourth panel from a first adjustable circumferential spacing based on a deformed shape-memory configuration of the shape-memory actuator, toward a second adjustable circumferential spacing, less than the first adjustable circumferential spacing, based on a recovered shape-memory configuration of the shape-memory actuator. For example, the panel portion 132 and the panel portion 134 can be drawn together by actuation of the adjustable fastener 136.

In some implementations, the shape-memory actuator can be mechanically deformable to have a first length in a deformed shape-memory configuration, and can be thermally recoverable to have a second length, different from the first length, in a recovered shape-memory configuration. In some implementations, actuating at least a portion of the shape-memory actuator to apply a second compressive force, greater than the first compressive force, about the body portion can include applying a predetermined temperature to the shape-memory actuator such that the shape-memory actuator is at least partly transitioned from the first length to the second length. For example, the panel portions 132 and 134 can be drawn apart to tension and deform the shape-memory material coil of the adjustable fastener 136, and the shape-memory coil can be heated to cause the coil to recover toward its shorter, more tightly coiled configuration.

In some implementations, the shape-memory actuator can include a metal alloy comprising nickel and titanium arranged as a helical tension spring configured to have the first length in the deformed shape-memory configuration and to have the second length in the recovered shape-memory configuration. For example, the adjustable fastener 136 is a coil of nickel and titanium alloy that can be mechanically stretched to a deformed configuration, and then heated to cause the alloy to recover to its original, more tightly coiled shape.

In some implementations, the process 900 can also include actuating at least a portion of the shape-memory actuator to at least partly release the second compressive force about the body portion. In some implementations, the process 900 can also include mechanically deforming the shape-memory actuator to the deformed shape-memory configuration, and at least partly releasing the second compressive force about the body portion. For example, the controller 190 can turn off the power used to heat and recover the shape-memory materials of the adjustable fasteners 136, allowing the adjustable fasteners to relax and/or be lengthened by tension across the active longitudinal panels 130a, 130b, increasing the diameter of the thigh portion 101a or the calf portion 101b and relieving a portion of the compression provided to the leg 10.

In some implementations, the process 900 can also include actuating another portion of the shape-memory actuator to apply a third compressive force, greater than the first compressive force, wherein the second compressive force is applied to a first region of the body portion and the third compressive force is applied to a second region of the body portion different from the first region. For example, the active subportion 710a of the example compressive garment 700 can be actuated separately from the active subportion 710b. As such the amount of compression provided to the region of the leg 10 near the active subportion 710a can be different from the amount of compression provided to the different region of the leg 10 near the active subportion 710b.

The applications for dynamic and controlled graded compression are extensive. Examples of such applications include a path for orthostatic intolerance, deep vein thrombosis (DVT) prevention and heart failure (HF) via electrocardiogram gating as a more dynamic treatment to compliment cardiac rehabilitation in this population. Each of these conditions represent groups with significant gaps exist for treatment options and prevention. While orthostatic intolerance and conditions such as POTS or Postural Orthostatic Tachycardia Syndrome are thought to influence up to 3 million Americans, often influencing younger women, other studies have found marked problems with positional hypotension in the elderly (>50% prevalence) with significant morbidity and mortality. Similarly, the prevalence of DVT clots has been reported to be 3-5% on longer duration airline flights, however there is significant postsurgical risk for clots that can remain for several days post hospitalization.

Previous work in the heart failure population using Enhanced External Counterpulsation (EECP), a form of gated compression performed with tethered pressure sleeves at rest over the legs in medical centers has resulted in marked improvements in some patients with some evidence for improved collateral circulation in the heart. Recently the National Institutes for Health has announced an RFA for novel enhancements or complimentary methods for cardiac rehabilitation in the HF population. Compression during diastole enhances venous return and cardiac filling and improves the benefits of the “muscle pumps” during exercise.

Heart failure is a major cause of hospitalizations in people above at 65 years of age, with an annual cost that has been estimated to be nearly $40 billion annually in the US. A downward spiral in activity remains a major contributor to morbidity; a gated and graded compression garment for this population could improve the benefits of rehabilitation and thus provide a practical, noninvasive therapy.

FIG. 10 is a schematic diagram of an example of a generic computer system 1000. The system 1000 can be used for the operations described in association with the process 900 according to one implementation. For example, the system 1000 may be included in the controller 190.

The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In one implementation, the memory 1020 is a computer-readable medium. In one implementation, the memory 1020 is a volatile memory unit. In another implementation, the memory 1020 is a non-volatile memory unit.

The storage device 1030 is capable of providing mass storage for the system 1000. In one implementation, the storage device 1030 is a computer-readable medium. In various different implementations, the storage device 1030 may be a floppy disk device, a hard disk device, an optical disk device, or a tape device.

The input/output device 1040 provides input/output operations for the system 1000. In one implementation, the input/output device 1040 includes a keyboard and/or pointing device. In another implementation, the input/output device 1040 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

In a first aspect, a tubular compressive garment includes a first longitudinal panel having a first panel portion, a second panel portion, and a first adjustable fastener configurable to connect the first panel portion to the second panel portion at a selected circumferential spacing, and a second longitudinal panel having a third panel portion, a fourth panel portion, and a second adjustable fastener comprising a shape-memory actuator configurable to connect the third panel portion to the fourth panel portion at a first adjustable circumferential spacing based on a deformed shape-memory configuration of the shape-memory actuator, and a second adjustable circumferential spacing , different from the first adjustable circumferential spacing, based on a recovered shape-memory configuration of the shape-memory actuator.

Various aspects can include some, all, or none of the following features. The shape-memory actuator can be a metal alloy comprising nickel and titanium, the shape-memory actuator being configured to have a first length in the deformed shape-memory configuration and to have a second length, different from the first length, in the recovered shape-memory configuration, wherein the metal alloy can be configured to be at least partly transitioned from the second length to the first length by tension between the third panel portion and the fourth panel portion, and wherein the metal alloy is configured to be at least partly transitioned from the first length to the second length by applying a predetermined temperature to the metal alloy. The metal alloy can be arranged as a helical tension spring and configured to be tensioned to the first length, and can be configured to at least partly return to the second length at the predetermined temperature, wherein the second length is shorter than the first length. The first adjustable fastener can be a lacing arranged between the first panel portion and the second panel portion. The second adjustable fastener can be a lacing comprising a lace arranged between the third panel portion and the fourth panel portion, wherein the lace comprises the shape-memory actuator. The lacing can include a thermally insulating boning configured to withstand a predetermined temperature of actuation of the shape-memory actuator. The second longitudinal panel can include at least one cross-fastener connecting the third panel portion to the fourth panel portion at no greater than the first adjustable circumferential spacing. The second longitudinal panel can include a fifth panel portion extending circumferentially between the third panel portion and the fourth panel portion. The tubular compressive garment can include a controller module configured to selectably actuate the shape-memory actuator.

In a second aspect, a method of providing compression includes adorning a body portion with a tubular compressive garment having a first panel portion, a second panel portion, and a first adjustable fastener configurable to connect the first panel portion to the second panel portion, a second longitudinal panel having a third panel portion, a fourth panel portion, a second adjustable fastener comprising a shape-memory actuator configurable to connect the third panel portion to the fourth panel portion, adjusting the first adjustable fastener to apply a first compressive force about the body portion, and actuating at least a portion of the shape-memory actuator to apply a second compressive force, greater than the first compressive force, about the body portion.

Various implementations can include some, all, or none of the following features. The method can also include actuating at least a portion of the shape-memory actuator to at least partly release the second compressive force about the body portion. Adjusting the first adjustable fastener to apply a first compressive force about the body portion can include adjusting the first adjustable fastener to draw the first panel portion toward the second panel portion at a selected circumferential spacing. The method can also include drawing the third panel portion and the fourth panel portion apart to a first adjustable circumferential spacing, and deforming the shape-memory actuator to a deformed shape-memory configuration. The method can also include limiting, by at least one cross-fastener connecting the third panel portion to the fourth panel portion, drawing of the third panel portion from the fourth panel portion to no greater than the first adjustable circumferential spacing. Actuating at least a portion of the shape-memory actuator to apply a second compressive force, greater than the first compressive force, about the body portion can include drawing, by the shape-memory actuator, the third panel portion toward the fourth panel portion from a first adjustable circumferential spacing based on a deformed shape-memory configuration of the shape-memory actuator, toward a second adjustable circumferential spacing, less than the first adjustable circumferential spacing, based on a recovered shape-memory configuration of the shape-memory actuator. The shape-memory actuator can be mechanically deformable to have a first length in a deformed shape-memory configuration, and can be thermally recoverable to have a second length, different from the first length, in a recovered shape-memory configuration. Actuating at least a portion of the shape-memory actuator to apply a second compressive force, greater than the first compressive force, about the body portion can include applying a predetermined temperature to the shape-memory actuator such that the shape-memory actuator is at least partly transitioned from the first length to the second length. The method can include mechanically deforming the shape-memory actuator to the deformed shape-memory configuration, and at least partly releasing the second compressive force about the body portion. The shape-memory actuator can include a metal alloy arranged as a helical tension spring configured to have the first length in the deformed shape-memory configuration and to have the second length in the recovered shape-memory configuration. The method can also include actuating another portion of the shape-memory actuator to apply a third compressive force, greater than the first compressive force, wherein the second compressive force is applied to a first region of the body portion and the third compressive force is applied to a second region of the body portion different from the first region.

In a third aspect, a system for applying compression to a body portion, the system including a tubular compressive garment having a first longitudinal panel having a first panel portion, a second panel portion, and a first adjustable fastener configurable to connect the first panel portion to the second panel portion at a selected circumferential spacing, and a second longitudinal panel having a third panel portion, a fourth panel portion, and a second adjustable fastener comprising a shape-memory actuator configurable to connect the third panel portion to the fourth panel portion at a first adjustable circumferential spacing based on a deformed shape-memory configuration of the shape-memory actuator, and a second adjustable circumferential spacing, different from the first adjustable circumferential spacing, based on a recovered shape-memory configuration of the shape-memory actuator, and a controller module configured to selectably actuate at least a portion of the shape-memory actuator.

The systems and techniques described here may provide one or more of the following advantages. First, a system can provide a controllably compressive garment that combines the controllability and ease of donning of an inflatable sleeve with the low mass and low profile of an elastic stocking. Second, the system can improve hemodynamics of the wearer. Third, the system can gate or time compression from body movements, body position, electrocardiogram heart cycle, or other biofeedback indicators. Fourth, the system can help the wearer maintain blood pressure with body position changes (e.g. Postural Tachycardia Syndrome, POTS, or the elderly). Fifth, the system can help the wearer counter conditions such as deep vein thrombosis (DVT) and chronic edema (fluid) conditions such as lymphedema and dependent edema. Sixth, the system can act as a cardiac assist device, as a treatment for heart failure patients going through cardiac rehabilitation. Seventh, the system can complement and accentuate natural physiological principals of the wearer, acting as a muscle pump to noninvasively improve the wearer's quality of life.

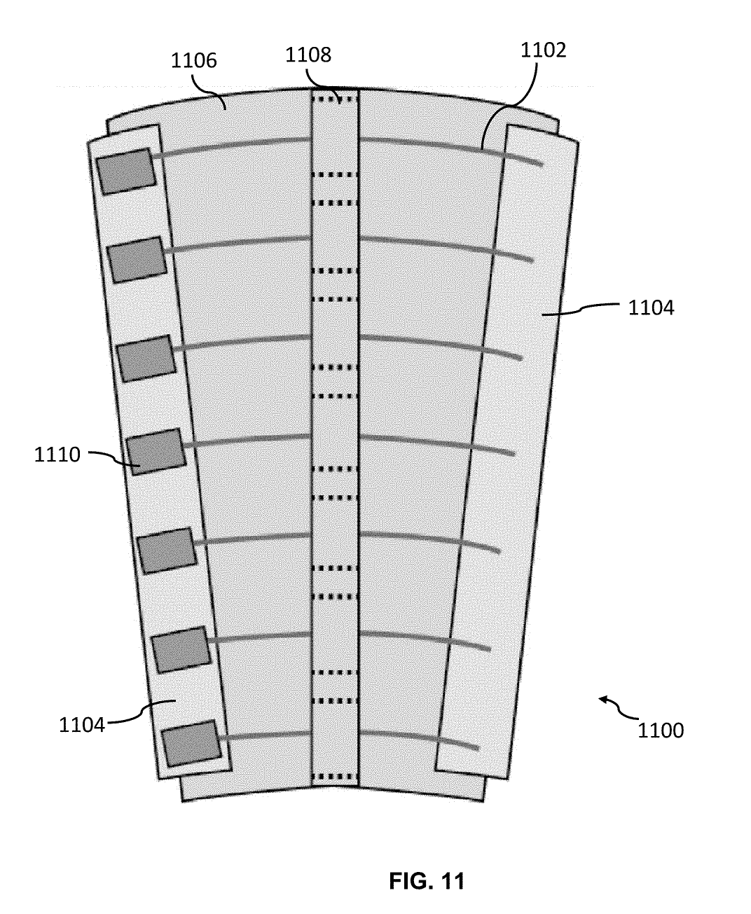

FIG. 11 is an opened view of an alternative embodiment similar to the open view of FIG. 3, but depicting a three-layer garment 1100. Garment 1100 includes seven superelastic links 1102 in the embodiment shown in FIG. 11, although in alternative embodiments there could be relatively more or fewer links 1102. Garment 1100 also includes a middle layer 1104, which defines a gap across which the links 1102 span. Middle layer 1104 is coupled to bottom layer 1106, such as by zippers, snaps, or other removable attachments as described above. Alternatively, in embodiments middle layer 1104 can be permanently affixed to bottom layer 1106. Tape 1108 is coupled to bottom layer 1106 and defines loops through which links 1102 can pass. Tape 1108 therefore prevents migration of links 1102 relative to the remainder of garment 1100 after expansion and/or contraction.

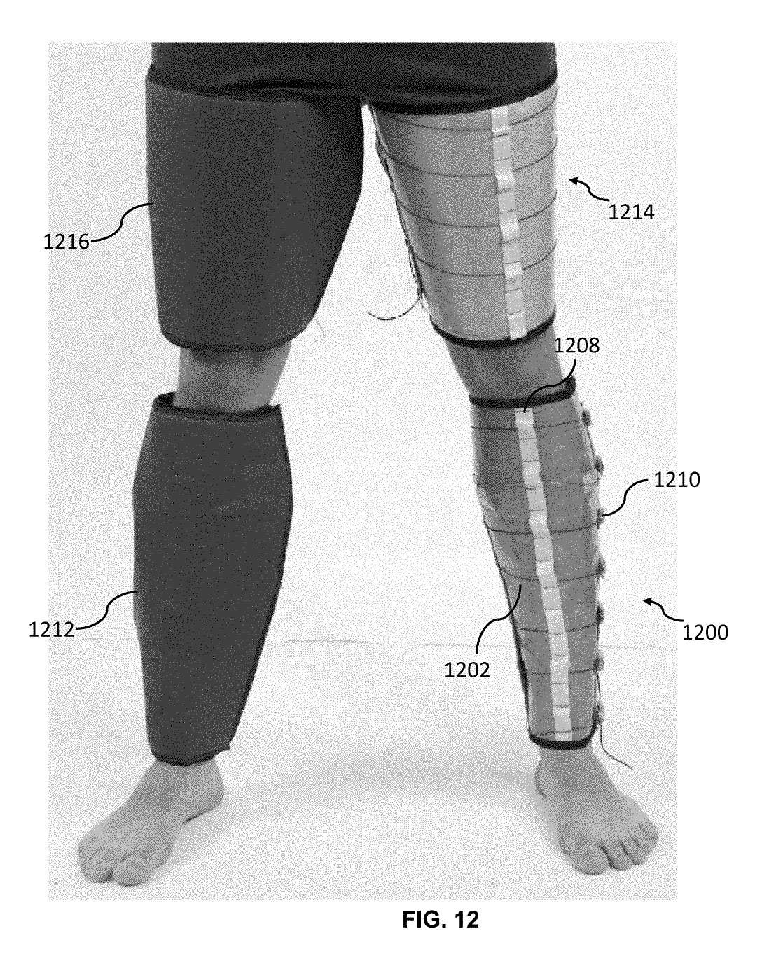

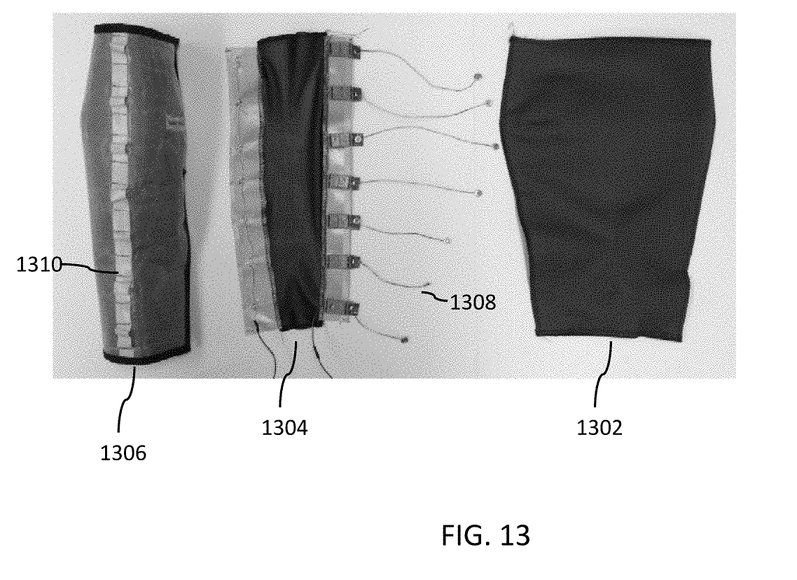

As links 1102 contract, the two portions of middle layer 1104 are pulled towards one another, narrowing the gap through which bottom layer 1106 is visible. This results in compression of the overall garment, as described above in more detail with respect to the other embodiments. For example, garment 1100 can be used as a compression garment for a calf. Garment 1100 can be paired with other compression garments that also contain superelastic components. For example, in an embodiment garment 1100 can be a compression garment for a calf and can be paired with a compression garment for a thigh, as depicted in FIG. 12. FIG. 11 also depicts tension circuit breakers 1110, described in more detail below with respect to FIGS. 13-15.

FIG. 12 depicts a system including four parts: left calf compression garment 1200, right calf compression garment 1212, left thigh compression garment 1214, and right thigh compression garment 1216. The four garments (1200, 1212, 1214, and 1216) can cooperate to provide compression therapy. In embodiments, the four garments (1200, 1212, 1214, and 1216) can be controlled by an external controller or power supply, either individually or separately. Left calf compression garment 1200 includes superelastic links 1202, which are similar to links 1102 previously described with respect to FIG. 11. Links 1202 are connected to circuit breakers 1210 to define a maximum tension level, as described in more detail below. Tape 1208 holds links 1202, similar to tape 1108 described above with respect to FIG. 11.

Garment 1200 is depicted with only the bottom and middle layers of the three-layer garment shown. In contrast, right calf compression garment 1212 shows only an outer layer. The outer layer shown for compression garment 1212 is a fabric that can be coupled to the middle and/or inner layers using hook and loop, snap, zipper, button, or other fastening mechanisms as described above. The outer layer protects the moving and powered members of the middle layer from external objects, and also protects the user from contact with moving or electrified components. Similarly, only middle and lower layers are shown with respect to left thigh compression garment 1214, while only the outer layer is visible in the right thigh compression garment 1216. It should be understood that a user could fully assemble the four garments (1200, 1212, 1214, and 1216) such that each contains a shape-memory compression middle layer surrounded by passive inner layers and outer layers.

FIG. 13 depicts an unfolded embodiment having three layers: outer layer 1302, middle layer 1304, and inner layer 1306. Outer layer 1302 is similar to the outer layers as shown in and described above with respect to FIG. 12. Middle layer 1304 includes a plurality of circuits 1308, each of which includes a shape memory segment and a tension-limited latch. In the view shown in FIG. 13, the shape memory segment components of the circuits 1308 are loose, though in operation the circuits would wrap around middle layer 1304 such that the shape memory segments are attached to the latches at both ends.

Inner layer 1306 protects the user from the heat generated by the shape memory elements in the circuits 1308 and helps distribute the force generated by circuits 1308 circumferentially. In embodiments, inner layer 1306 can be made primarily of polytetrafluoroethylene (PTFE) sheets to provide a heat resistant surface that allows for the actuators to compress easily. A strip of fiberglass ribbon tape 1310 is sewn down vertically in the center of inner layer 1308, with several open channels to route the circuits 408 around the anterior side of the inner layer 1306 and prevent movement of the circuits 1308 relative inner layer 1306.

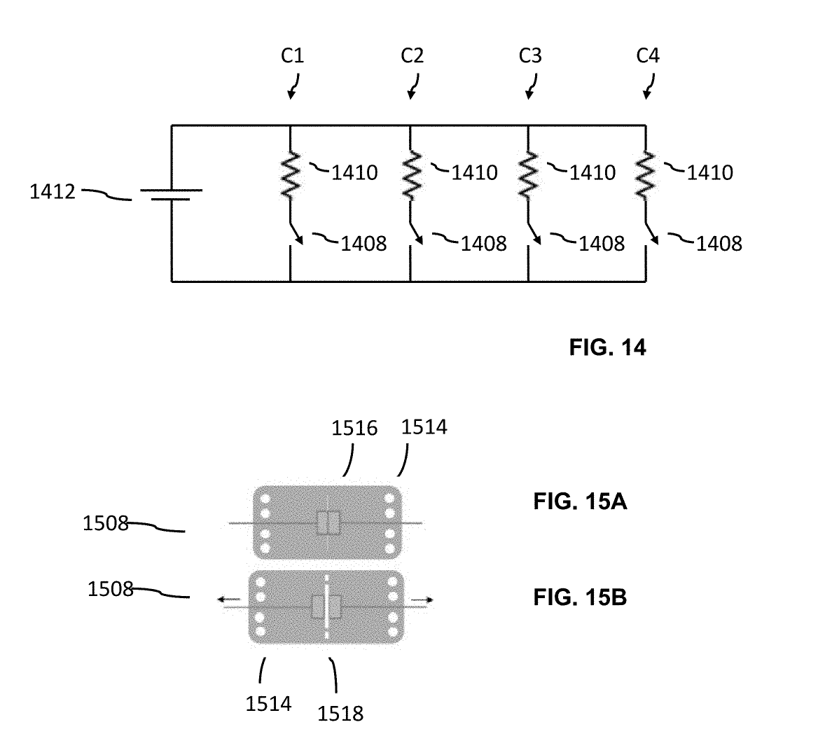

FIG. 14 is a circuit diagram corresponding to a compression garment, according to an embodiment. The circuit diagram depicted in FIG. 14 includes four coils, C1-C4. Each coil C1-C4 includes a switch 1408 and a resistor 1410. The entire system, including all of the coils C1-C4, is powered by voltage source 1412.

Each coil C1-C4 includes a switch 1408, which is a schematic representation of a tension-based latch. If the first coil C1 is under sufficient tension, the switch 1408 of that coil C1 opens, such that no current flows through coil C1. Power from voltage source 1412 nonetheless remains available to the other coils C2-C4. Each of the switches 1408 can operate independently based upon tension at a different portion of a garment, in embodiments.

Resistors 1410 dissipate energy to cause contraction. In the examples described above, resistors 1410 can be superelastic. Current through resistors 1410 causes heating, which activates the thermomechanical response of the resistors 1410 to cause both heating and corresponding contraction. Contraction of resistors 1410 can cause tension on the corresponding latches (i.e., switches 1408).

FIGS. 15A and 615 depict a latch 1508, according to an embodiment. Latch 1508 includes wire 1514, which can form a loop around a garment as described in more detail above. Wire 1514 can also be coupled to a voltage source (e.g., voltage supply 1412 of FIG. 14), which is not shown in FIGS. 15A and 15B. Wire 1514 is connected to contacts 1516. In FIG. 15A, contacts are touching one another, closing the circuit such that power flows through wire 1514. As current flows through wire 1514, it is heated and undergoes a transition to shrink or otherwise deform, increasing the tension on latch 1508. Once the tension on latch 1508 reaches some threshold, latch 1508 can expand such that contacts 1516 are separated to define a gap 1518. Once gap 1518 is formed, current cannot flow from one side of latch 1508 to the other, and in normal operating conditions the temperature of wire 1514 will begin to drop as current from the voltage supply no longer flows through it. Thus, latch 1508 of FIGS. 15A and 15B is a self-contained, independent control system that maintains a desired level of tension on a compression garment without requiring monitoring, communication, and processing systems. Alternative mechanisms for tension-based circuit breaking can include springs with predetermined, maximum force before compression, elastically deformable clamps, or other systems that will break apart to open the electric circuit upon application of sufficient force.

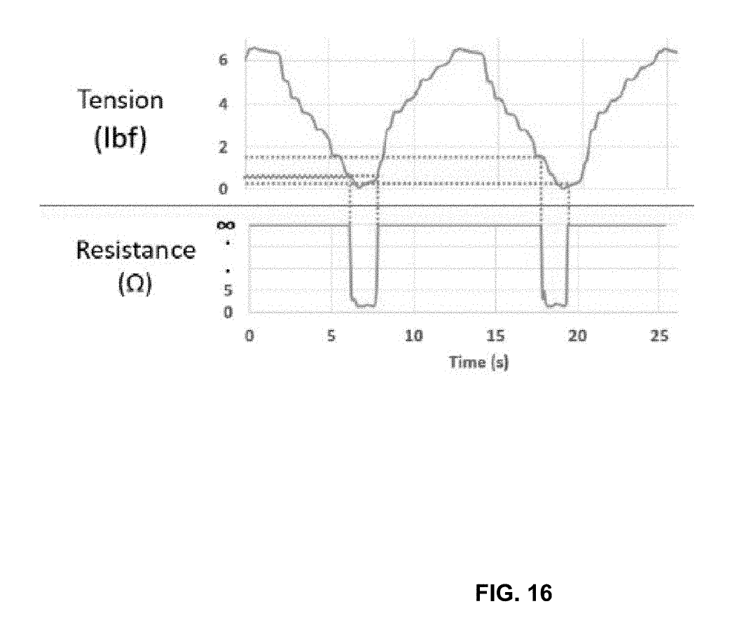

FIG. 16 depicts a chart of tension in a garment (top) and resistance of a superelastic coil (bottom) for the garment depicted above in FIG. 12. At high tension, the resistance is high (due to the opening of the circuit breakers). In contrast, below a threshold of about 1.8 lbf (shown with dashed lines) to about 0 lbf, resistance drops to near zero as the tension-based circuit breakers close. In alternative embodiments, the tension levels that cause opening or closing of the circuit breakers can be varied. The level of tension required to open or close the circuit breakers can be determined based upon a desired maximum level of compression in the garment.

Although a few implementations have been described in detail above, other modifications are possible. For example, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A system of compression garments comprising:
a left calf portion comprising:
a first inner layer;
a first middle layer coupled to the first inner layer, the first middle layer comprising a first plurality of circuits, each circuit of the first plurality of circuits comprising a superelastic cable and a circuit breaker;
a first outer layer removably coupled to at least one of the first inner layer and the first middle layer;
a right calf portion comprising:
a second inner layer;
a second middle layer coupled to the second inner layer, the second middle layer comprising a second plurality of circuits, each circuit of the second plurality of circuits comprising a superelastic cable and a circuit breaker;
a second outer layer removably coupled to at least one of the second inner layer and the second middle layer;
a left thigh portion comprising:
a third inner layer;
a third middle layer coupled to the third inner layer, the third middle layer comprising a third plurality of circuits, each circuit of the third plurality of circuits comprising a superelastic cable and a circuit breaker;
a third outer layer removably coupled to at least one of the third inner layer and the third middle layer; and
a right thigh portion comprising:
a fourth inner layer;
a fourth middle layer coupled to the fourth inner layer, the fourth middle layer comprising a fourth plurality of circuits, each circuit of the fourth plurality of circuits comprising a superelastic cable and a circuit breaker;
a fourth outer layer removably coupled to at least one of the fourth inner layer and the fourth middle layer.

2. The system of compression garments of claim 1, wherein the superelastic cables is a metal alloy comprising nickel and titanium configured to have a first length in the deformed shape-memory configuration and to have a second length, different from the first length, in the recovered shape-memory configuration, wherein the metal alloy is configured to be at least partly transitioned from the second length to the first length by tension between the third panel portion and the fourth panel portion, and wherein the metal alloy is configured to be at least partly transitioned from the first length to the second length by applying a predetermined temperature to the metal alloy.

3. The system of compression garments of claim 2, wherein the metal alloy is arranged as a cable and is configured to be tensioned to the first length, and configured to at least partly return to the second length at the predetermined temperature, wherein the second length is shorter than the first length.

4. The system of compression garments of claim 1, wherein each of the left calf portion, the right calf portion, the left thigh portion, and the right thigh portion further comprise a tape coupled to the respective inner layer, and each of the tapes is configured to hold the superelastic cables of it respective middle layer.

5. A compression garment comprising:
an inner layer;
a middle layer coupled to the inner layer, the middle layer comprising a plurality of circuits, each circuit of the plurality of circuits comprising a superelastic cable and a circuit breaker; and
a outer layer removably coupled to at least one of the inner layer and the middle layer.

6. The compression garment of claim 5, wherein the compression garment is configured for use on a calf and comprises seven circuits.

7. The compression garment of claim 5, wherein the compression garment is configured for use on a thigh and comprises five circuits.

8. The compression garment of claim 5, further comprising a tape coupled to the inner layer and configured to hold each of the superelastic cables.

9. The compression garment of claim 5, wherein the circuit breakers are configured to change the circuit between a closed configuration and an open configuration at a predetermined tension level.

10. A method of providing compression comprising:
adorning a body portion with a tubular compressive garment comprising:
an inner layer;
a middle layer coupled to the inner layer, the middle layer comprising a plurality of circuits, each circuit of the plurality of circuits comprising a superelastic cable and a circuit breaker; and
a outer layer removably coupled to at least one of the inner layer and the middle layer;
adjusting the garment to apply a first compressive force about the body portion; and
actuating at least one of the superelastic cables to apply a second compressive force, greater than the first compressive force, about the body portion.

11. The method of claim 10, wherein adjusting the garment to apply a first compressive force about the body portion comprises adjusting a first adjustable fastener to draw a first panel portion toward a second panel portion at a selected circumferential spacing.

12. The method of claim 11, further comprising drawing a third panel portion and a fourth panel portion apart to a first adjustable circumferential spacing, and deforming the at least one of the superelastic cables to a deformed shape-memory configuration.

13. The method of claim 10, wherein each of the superelastic cables is mechanically deformable to have a first length in a deformed shape-memory configuration, and is thermally recoverable to have a second length, different from the first length, in a recovered shape-memory configuration.
